(19) United States
(12) Patent Application Publication
(10) Pub. No.: US 2025/0205451 A1
(43) Pub. Date: Jun. 26, 2025

(54) COCHLEAR IMPLANTABLE CATHETER AND CELL DELIVERY DEVICE THEREOF

(71) Applicant: National Defense Medical Center, Taipei City (TW)

(72) Inventor: Chih-Hung Wang, New Taipei City (TW)

(21) Appl. No.: 18/950,273

(22) Filed: Nov. 18, 2024

(30) Foreign Application Priority Data
Dec. 20, 2023 (TW) 112149634

(51) Int. Cl. A61M 25/00

(52) U.S. Cl. CPC A61M 25/0067

(57) ABSTRACT

A cochlear implantable catheter and a cell delivery device thereof are provided. The cochlear implantable catheter includes an implantable catheter body, and a proximal end and a leading tip are defined at two opposite ends of the implantable catheter body. A central lumen is formed at a center of the implantable catheter body. An outer surface of the implantable catheter body has at least one guide groove that extends in a longitudinal direction of the implantable catheter body and a plurality of lateral through holes that penetrate from the outer surface of the implantable catheter body to the central lumen. The implantable catheter body is configured to penetrate into a cochlea of an inner ear of a patient, and is bendable along cochlear turns of the patient, such that cells or medications can enter the cochlea of the patient through the central lumen and the lateral through holes.

### CROSS-REFERENCE TO RELATED PATENT APPLICATION

This application claims the benefit of priority to Taiwan Patent Application No. 112149634, filed on Dec. 20, 2023. The entire content of the above identified application is incorporated herein by reference.

Some references, which may include patents, patent applications and various publications, may be cited and discussed in the description of this disclosure. The citation and/or discussion of such references is provided merely to clarify the description of the present disclosure and is not an admission that any such reference is “prior art” to the disclosure described herein. All references cited and discussed in this specification are incorporated herein by reference in their entireties and to the same extent as if each reference was individually incorporated by reference.

### FIELD OF THE DISCLOSURE

The present disclosure relates to a cochlear implantable catheter and a cell delivery device, and more particularly to a cochlear implantable catheter and a cell delivery device for delivering cells or medications into an inner ear of a human body.

### BACKGROUND OF THE DISCLOSURE

An auditory receptor of an inner ear (i.e., a cochlea) and an auditory nerve are important structures of a human body for receiving external sound signals, and can easily lose their function due to drugs, surroundings, aging, or mutation. The hearing function of an individual is likely to suffer permanent damage and becomes unrecoverable once impaired, and this is the bottleneck of current treatments for hearing loss. However, the advancement of gene and regenerative medicine facilitates understanding of a gene transfer therapy for hearing loss and cochlear hair cell regeneration, thereby raising the expectation to treat hearing loss with a cell therapy.

The cell therapy for treating hearing loss is mainly to inject cells or medications into the inner ear. Accordingly, the cells injected into the inner ear can act as a replacement or regenerate the required auditory hair cells, or the implanted cellular medications can enable original cells of the inner ear to be differentiated or transformed into the required auditory hair cells, such that the hearing of a patient can be recovered. In the cell therapy, the injected cells can be divided into autologous cells and allogeneic cells according to cell sources, or can be divided into progenitor cells and stem cells according to cell types. The used medications can be cellular growth factors that promote growth or transformation of the cells, or can be small molecule drugs.

How to effectively inject the cells or the medications into the cochlea is a critical factor in the cell therapy for treating hearing loss. While an outer structure of cochlear turns of the cochlea is formed by the bony labyrinth, a cavity of the cochlear turns (from a basal turn to an apical turn) is formed by tiny tubes having a spiral shape, and scala media, scala vestibuli, and scala tympani are included therein. The cavity of the cochlear turns is an enclosed structure filled with lymph. The volume of the human cochlea is only about 10 μl. Due to lymphatic pressure and obstruction of a round window membrane, partial extravasation of the medications or the cells is likely to occur, thereby reducing the efficiency of cochlear drug or cell delivery.

In the conventional technology, an implant electrode of a cochlear implant is used as an implant catheter. Apart from an implant electrode array and conductive wires, the implant catheter further includes a lumen for loading and delivering the medications to the cochlea of a patient. Since said catheter is installed with electrodes and the conductive wires for stimulation of the auditory nerve, and is additionally installed with the lumen for loading or flowing of the medications, it is necessary to increase a diameter of the implant catheter. Furthermore, the implant catheter needs to be implanted together with the electrodes, and is thus a permanent implant device. However, the design of the present disclosure is applicable for a single therapy session in delivering the medications or the cells to the cochlea.

Therefore, in the technology of attempting to directly and partially deliver the cells or the medications to the cochlea, considerable challenges remain to be addressed. As such, how to improve the structural design of a device, so as to overcome the above-mentioned practical deficiencies, has become one of the important issues to be solved in the treatment of cochlear cells.

### SUMMARY OF THE DISCLOSURE

In response to the technical inadequacies in partial delivery of cells or medications to a cochlea, the present disclosure provides a cochlear implantable catheter and a cell delivery device thereof.

In order to solve the above-mentioned problems, one of the technical aspects adopted by the present disclosure is to provide a cochlear implantable catheter, which is configured to deliver cells or medications to a cochlea of a patient. The cochlear implantable catheter includes an implantable catheter body. The implantable catheter body is flexible, and a proximal end and a leading tip are defined at two opposite ends of the implantable catheter body. A portion of the implantable catheter body that is implanted into the cochlea of the patient is defined as an implant section, the implantable catheter body has a connection end that is disposed at one side of the implant section toward the proximal end, a central lumen that penetrates from a center of the proximal end to the leading tip is formed inside the implantable catheter body, and the implant section has at least one guide groove formed on an outer surface thereof and a plurality of lateral through holes that penetrate from the outer surface of the implant section to the central lumen. The implantable catheter body is configured to penetrate into the cochlea of the patient by orienting the leading tip toward the cochlea of the patient, and is capable of bending and moving forward along a spiral structure of the cochlea of the patient, so as to enter between a basal turn and an apical turn of the cochlea of the patient. The cells or the medications are capable of being guided into the central lumen from the proximal end and entering the cochlea through the central lumen and the plurality of lateral through holes.

In order to solve the above-mentioned problems, another one of the technical aspects adopted by the present disclosure is to provide a cell delivery device. The cell delivery device includes the above-mentioned cochlear implantable catheter and a delivery module. The delivery module is connected to the cochlear implantable catheter, and is configured to deliver cells or medications to a cochlea. The delivery module includes a pressure generator and a connection tube connected to an outlet of the pressure generator. A fluid that contains the cells or the medications is stored in the pressure generator, and the pressure generator drives and outputs the fluid that contains the cells or the medications from the outlet to the connection tube. One end of the connection tube that is away from the pressure generator is connected to the proximal end of the implantable catheter body, such that the cells or the medications are capable of entering the central lumen of the implantable catheter body and entering the cochlea of the patient through the central lumen and the plurality of lateral through holes.

Therefore, in the cochlear implantable catheter and the cell delivery device provided by the present disclosure, since the cochlear implantable catheter has good bendability, the leading tip of the cochlear implantable catheter can bend and enter deep into the cochlea in a spiral manner (along a spiral ligament on an outer wall of a cochlear cavity and toward the apical turn) when the leading tip of the cochlear implantable catheter is implanted into the basal turn of the cochlear of the patient. Through the design of the guide groove, resistance from an outer wall of the implantable catheter body and lymph in the cochlea can be decreased, thereby increasing an implant success rate of the cochlear implantable catheter and reducing implant damage incurred by a catheter to a main body of the cochlea.

Furthermore, since the cochlear implantable catheter of the present disclosure is designed to have the guide groove and the lateral through holes that are evenly arranged on the implantable catheter body, the cells or the medications can be guided to be evenly distributed and released to different positions of the cochlea, and lymphatic extravasation of the cochlea can be prevented.

These and other aspects of the present disclosure will become apparent from the following description of the embodiment taken in conjunction with the following drawings and their captions, although variations and modifications therein may be affected without departing from the spirit and scope of the novel concepts of the disclosure.

### BRIEF DESCRIPTION OF THE DRAWINGS

The described embodiments may be better understood by reference to the following description and the accompanying drawings, in which:

FIG. 1 is a schematic view of a cochlear implantable catheter according to a first embodiment of the present disclosure;

FIG. 2 is a schematic cross-sectional view of the cochlear implantable catheter according to the present disclosure;

FIG. 3 is a partial schematic enlarged view of the cochlear implantable catheter taken from part III of FIG. 1;

FIG. 4 is a partial schematic enlarged view of another configuration of the cochlear implantable catheter according to the present disclosure;

FIG. 5 is a schematic view of a cell delivery device according to one embodiment of the present disclosure;

FIG. 6 is a schematic view of the cell delivery device according to another embodiment of the present disclosure;

FIG. 7 is a schematic view showing an operation process of implanting the cochlear implantable catheter into a cochlea of a patient for delivery of cells or medications to treat hearing loss according to the present disclosure;

FIG. 8 is a schematic enlarged view showing the cochlear implantable catheter delivering the cells or the medications into the cochlea of the patient according to the present disclosure;

FIG. 9 is a schematic view of the cochlear implantable catheter according to a second embodiment of the present disclosure; and

FIG. 10 is a schematic cross-sectional view of the cochlear implantable catheter according to the second embodiment of the present disclosure.

### DETAILED DESCRIPTION OF THE EXEMPLARY EMBODIMENTS

The present disclosure is more particularly described in the following examples that are intended as illustrative only since numerous modifications and variations therein will be apparent to those skilled in the art. Like numbers in the drawings indicate like components throughout the views. As used in the description herein and throughout the claims that follow, unless the context clearly dictates otherwise, the meaning of “a,” “an” and “the” includes plural reference, and the meaning of “in” includes “in” and “on.” Titles or subtitles can be used herein for the convenience of a reader, which shall have no influence on the scope of the present disclosure.

The terms used herein generally have their ordinary meanings in the art. In the case of conflict, the present document, including any definitions given herein, will prevail. The same thing can be expressed in more than one way. Alternative language and synonyms can be used for any term(s) discussed herein, and no special significance is to be placed upon whether a term is elaborated or discussed herein. A recital of one or more synonyms does not exclude the use of other synonyms. The use of examples anywhere in this specification including examples of any terms is illustrative only, and in no way limits the scope and meaning of the present disclosure or of any exemplified term. Likewise, the present disclosure is not limited to various embodiments given herein. Numbering terms such as “first,” “second” or “third” can be used to describe various components, signals or the like, which are for distinguishing one component/signal from another one only, and are not intended to, nor should be construed to impose any substantive limitations on the components, signals or the like.

### First Embodiment

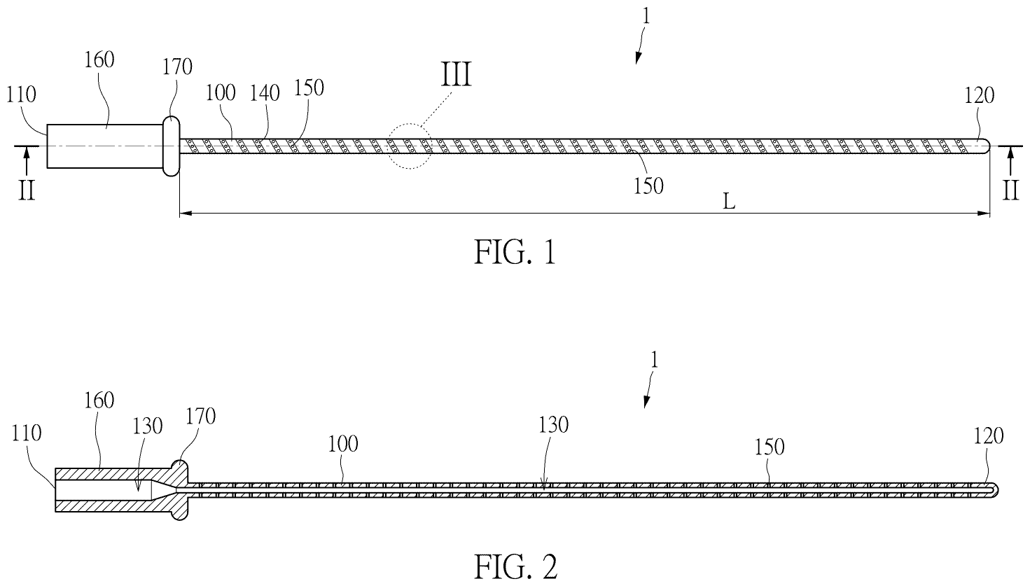

Referring to FIG. 1 to FIG. 8, one embodiment of the present disclosure provides a cochlear implantable catheter and a cell delivery device that are configured to deliver cells or medications into a cochlea of a patient, so as to treat hearing loss of the patient by cell, medication, or gene therapy. It should be noted that, in the present disclosure, the cells or the medications for treating hearing loss include: cells such as autologous cells, stem cells, and progenitor cells and vehicles for carrying the cells, genes used to promote growth of auditory hair cells or transformation of the cells into the auditory hair cells (which include deoxyribonucleic acid (DNA), plasmid DNA, small interfering ribonucleic acid (siRNA), and oligonucleotides) and delivery vehicles thereof, or medications such as cellular growth factors, regenerative drugs, exosomes, or combinations thereof.

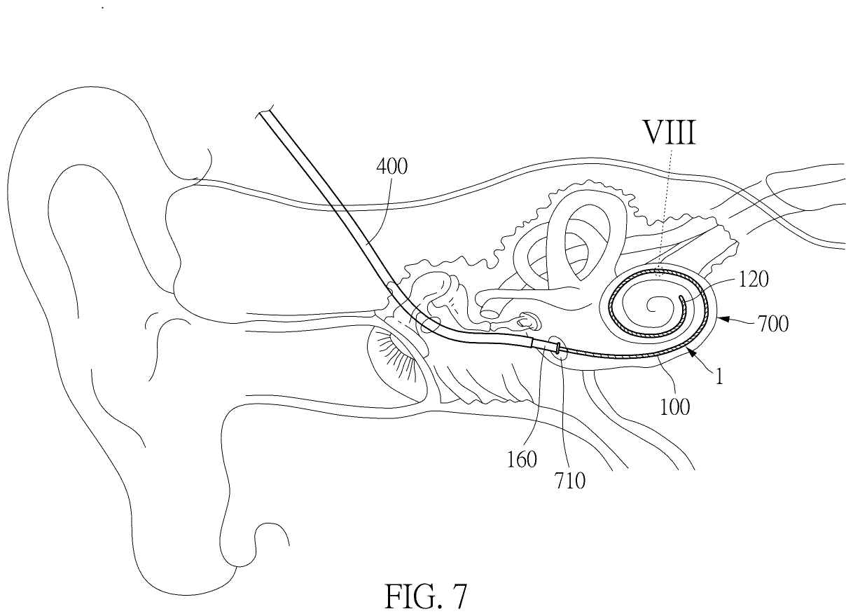

As shown in FIG. 1, FIG. 2, and FIG. 7, a cochlear implantable catheter 1 of the present disclosure is configured to guide the cells or the medications for treating hearing loss into a cochlea 700 of the patient. In the present embodiment, the cochlear implantable catheter 1 includes an implantable catheter body 100. In one exemplary embodiment, the implantable catheter body 100 is formed by a suitable biocompatible material. In one embodiment, the material of the implantable catheter body 100 is silicone (e.g., SILASTIC® MDX 4-4210). In another embodiment, the implantable catheter body 100 is formed by fluororesin, polyurethane, polyvinyl chloride, or similar materials.

The structure of the implantable catheter body 100 is substantially a slender tubular body with flexibility, and the implantable catheter body 100 can define a proximal end 110 and a leading tip 120 that are opposite to each other. The implantable catheter body 100 can be implanted into the cochlea of the patient by orienting the leading tip 120 toward the cochlea of the patient, and a portion of the implantable catheter body 100 that is implanted into the cochlea of the patient is defined as an implant section. The implantable catheter body 100 has a connection end 160 at one side toward the proximal end 110, and the connection end 160 is configured to connect with a catheter for delivering the cells or the medications. A protruding ring 170 is further disposed at a junction of the connection end 160 and the implant section of the implantable catheter body 100, and is provided for a user to identify a range of the implant section of the implantable catheter body 100. A diameter of the protruding ring 170 is configured to be greater than a diameter of the implant section and a diameter of the connection end 160. Accordingly, an operator can clearly identify the position of the protruding ring 170, and an implant length can be prevented from exceeding a length L of the implant section when the operator implants the implantable catheter body 100 into the cochlea of the patient.

The implant section of the implantable catheter body 100 has a central axial line along the implantable catheter body 100, a central lumen 130 that penetrates from a center of the connection end 160 and the proximal end 110 to a center of the leading tip 120, at least one guide groove 140 that has a spiral shape and surrounds an outer surface of the implantable catheter body 100 in 360 degrees, and a plurality of lateral through holes 150 that penetrate from an outer surface of the implant section of the implantable catheter body 100 to the central lumen 130.

The shape and the size of the implantable catheter body 100 are designed according to the structure of the human cochlea. Generally, a length of the cochlea of an adult is 30 mm. In one exemplary embodiment of the present disclosure, the length L of the implant section of the implantable catheter body 100 is configured to range between 8 mm and 30 mm. Hence, after the implantable catheter body 100 is implanted into the cochlea of the patient, a depth by which the leading tip 120 enters the cochlea is close to the 8 mm to 30 mm range, thereby allowing the implant section to be implanted at different positions (from a basal turn to an apical turn) of the cochlea of the patient.

In order to reduce expelling pressure applied to lymph in the cochlea when the implantable catheter body 100 is implanted into the cochlea of the patient, the diameter of the implant section of the implantable catheter body 100 is configured to range between 0.3 mm and 1.0 mm. In one exemplary embodiment, the diameter of the implant section is configured to range between 0.5 mm and 0.8 mm.

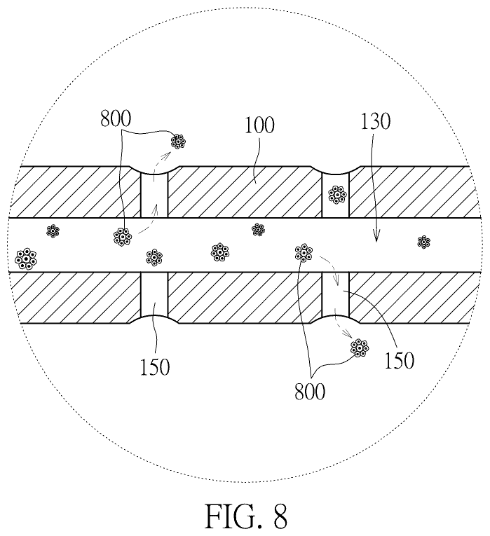

As shown in FIG. 8, the cells or the medications for treating hearing loss enters the central lumen 130 of the implant section from the central lumen 130 of the connection end 160, and flows to a tail end of the central lumen 130 of the leading tip 120 before being discharged from the lateral through holes 150.

In response to enlargement of a diameter of the central lumen 130, a sectional area of the central lumen 130 is increased, and a wall thickness of the implantable catheter body 100 is decreased at the same time. As such, the diameter of the central lumen 130 is to be adjusted according to a flow rate and a viscosity of a fluid delivered by the implantable catheter body 100, the required strength of the implantable catheter body 100, and other factors.

Specifically, the diameter of the central lumen 130 of the cochlear implantable catheter 1 and a diameter of the lateral through holes 150 can be changed according to the type of the cells or the medications to be delivered by the cochlear implantable catheter 1. For example, in one embodiment of the present disclosure, the cochlear implantable catheter 1 is used to load hearing regenerative drugs that contain cell spheroids. The cell spheroids contained in the hearing regenerative drugs are substantially formed by a combination of the progenitor cells and the stem cells with a cell quantity of from 2 to 10. An average diameter of each cell spheroid ranges between 30 μm and 100 μm. In the present embodiment, the diameter of the central lumen 130 is configured to range between 300 μm and 500 μm, and the diameter of the lateral through holes 150 is configured to range between 50 μm and 300 μm.

Through the above-mentioned configuration, the diameter of the central lumen 130 and the diameter of the lateral through holes 150 can cooperate with the size of the cell spheroids contained in the regenerative drugs delivered by the implantable catheter body 100, such that the medications and the cell spheroids can enter the cochlea of the patient by smoothly passing through the central lumen 130 and the lateral through holes 150.

In another embodiment of the present disclosure, the cochlear implantable catheter 1 is used to load the hearing regenerative drugs that contain a singular stem cell or inner ear hair cell or the hearing regenerative drugs that contain the cellular growth factors. Here, an average diameter of the stem cell or the inner ear hair cell ranges between 5 μm and 10 μm. The types of the cellular growth factors include various small molecule polypeptides and proteins, and an average diameter of the cellular growth factors ranges between 1 μm and 5 μm.

In the present embodiment, the diameter of the central lumen 130 is configured to range between 100 μm and 200 μm for cooperation with the size of a singular cell or growth factor, and the diameter of the lateral through holes 150 is configured to range between 10 μm and 50 μm. Through the above-mentioned configuration, the diameter of the central lumen 130 and the diameter of the lateral through holes 150 can cooperate with the size of the cell or the growth factor contained in the medications delivered by the implantable catheter body 100, such that the medications and the cells can enter the cochlea of the patient by smoothly passing through the central lumen 130 and the lateral through holes 150.

In the present embodiment, the at least one guide groove 140 is formed on the outer surface of the implantable catheter body 100 along a spiral path that extends in a longitudinal direction of the implantable catheter body 100. On one hand, the function of the at least one guide groove 140 is to increase bending elasticity of the implantable catheter body 100. When being implanted into a cochlear duct of the patient, the implantable catheter body 100 is easily bent and can spirally enter the apical turn of cochlear turns along a spiral ligament on an outer wall of a cochlear cavity.

On the other hand, a flow-guiding effect can be generated by the at least one guide groove 140. When the implantable catheter body 100 is implanted into the cochlear duct of the patient, resistance generated through contact between the outer surface of the implantable catheter body 100 and the lymph in the cochlear duct can be decreased, and an amplitude of lymphatic pressure increase in the cochlear cavity can be reduced. In this way, extravasation of the lymph in the cochlear cavity caused by an overly large pressure within the cochlea is less likely to occur.

In order to achieve the above-mentioned purposes, in the present embodiment, a helix angle of the at least one guide groove 140 is configured to range between 15 degrees and 60 degrees, and a width of the at least one guide groove 140 is configured to range between 10 μm and 200 μm. It should be noted that, when the helix angle of the at least one guide groove 140 (which is spiral-shaped) is 45 degrees, a shear stress generated through contact between the outer surface of the implantable catheter body 100 and the lymph reaches its minimum, thereby allowing an effect of reducing lymphatic extravasation to be improved.

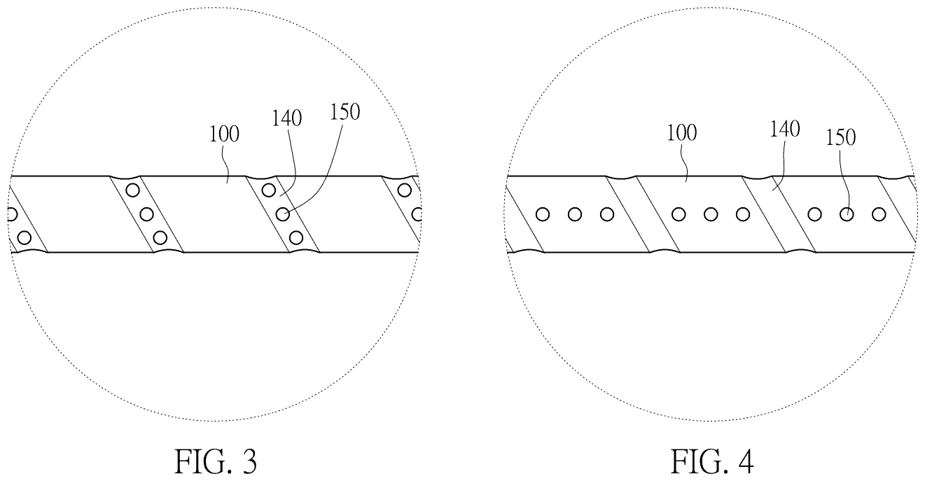

As shown in FIG. 3, in one embodiment of the present disclosure, the lateral through holes 150 are equidistantly arranged on the at least one guide groove 140 along an extension path of the at least one guide groove 140. In addition, a distance between two adjacent ones of the lateral through holes 150 is not less than 50 μm. In the present embodiment, since the lateral through holes 150 are arranged within the at least one guide groove 140, the cells or the medications that flow out of the lateral through holes 150 can enter and be guided by the at least one guide groove 140. Hence, the cells or the medications can be more evenly distributed within the cochlea.

As shown in FIG. 4, in another embodiment of the present disclosure, the lateral through holes 105 are arranged on the implantable catheter body 100 at positions that do not overlap with the at least one guide groove 140. That is, in present embodiment, the lateral through holes 105 are arranged on areas of the implantable catheter body 100 that do not have the at least one guide groove 140.

It should be noted that, in the design of the cochlear implantable catheter 1 of the present disclosure, the implantable catheter body 100 can simply act as a delivery catheter for delivering the fluid that contains the cells for treating hearing loss or the regenerative drugs. As such, installation of electrodes and wires of a cochlear implant is not required in the implantable catheter body 100, so as to prevent a diameter of the implantable catheter body 100 from being increased due to the electrodes or the wires. Furthermore, on the premise of ensuring that the implantable catheter body 100 has a sufficient strength to bear the resistance for being implanted into the cochlea, the wall thickness of the implantable catheter body 100 is to be decreased as much as possible. Based on the above, the diameter and a volume of the implantable catheter body 100 can be decreased, such that the lymphatic extravasation (caused by expelling of the lymph in the cochlea when the implantable catheter body 100 is implanted into the cochlea of the patient) is less likely to occur.

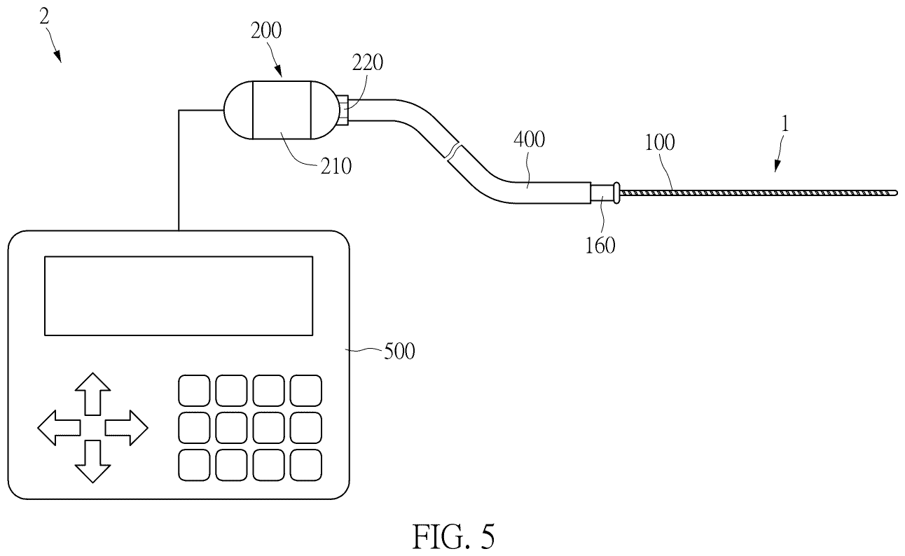

FIG. 5 shows a cell delivery device 2 that incorporates the cochlear implantable catheter 1 according to one embodiment of the present disclosure. The cell delivery device 2 includes the cochlear implantable catheter 1 and a delivery module 200 that is connected to the cochlear implantable catheter 1. The delivery module 200 includes a pressure generator 210, a connection tube 400 that is connected to an outlet 220 of the pressure generator 210, and a control device 500 that is connected to the pressure generator 210.

In the present embodiment, the pressure generator 210 can be selected from a micro pump, a peristaltic pump, a compressor, or other pressure generating devices. The control device 500 is connected to the pressure generator 210 for controlling actions of the pressure generator 210 and a flow velocity and a pressure of a fluid output by the pressure generator 210.

The operator pre-fills the central lumen 130 of the cochlear implantable catheter 1 with the medications or the delivery vehicles that contain the cells, and connects the connection end 160 of the cochlear implantable catheter 1 to the connection tube 400. After the implant section of the implantable catheter body 100 of the cochlear implantable catheter 1 is implanted into the cochlea of the patient, the cells or the medications within the cochlear implantable catheter 1 can be delivered into the cochlea of the patient through a pressure generated by the pressure generator 210.

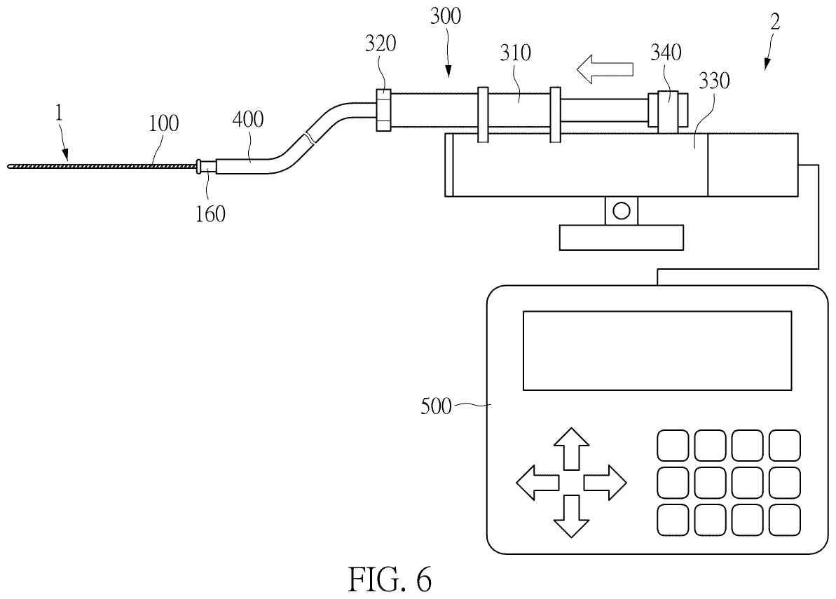

FIG. 6 shows the cell delivery device 2 that incorporates the cochlear implantable catheter 1 according to another embodiment of the present disclosure. It should be noted that the present embodiment is similar to the embodiment shown in FIG. 5, and the similarities therebetween will not be reiterated herein.

In the present embodiment, the cell delivery device 2 includes the cochlear implantable catheter 1 and a delivery module 300 that is connected to the cochlear implantable catheter 1. The delivery module 300 includes a pressure generator 310. The pressure generator 310 is a syringe, and a plunger (not shown in the figure) is disposed in the pressure generator 310. An outlet 320 is disposed at one end of the pressure generator 310, and the outlet 320 is connected to the connection tube 400. Another end of the pressure generator 310 that is opposite to the outlet 320 is connected to a linkage mechanism 340. The linkage mechanism 340 is connected to the plunger inside the pressure generator 310 and can be driven by a driving module 330, thereby enabling the plunger inside the pressure generator 310 to generate a pressure. The driving module 330 can be a driving device that is controlled by a stepper motor, so as to accurately control actions of the linkage mechanism 340. In this way, the purpose of accurately controlling a flow rate and a flow velocity of the output cells or medications can be achieved.

FIG. 7 and FIG. 8 are schematic views showing a process of implanting the cochlear implantable catheter 1 into the cochlea 700 of the patient and delivering the fluid that contains the cells or the medications for treating hearing loss into the cochlea 700 of the patient. As shown in FIG. 7, by orienting the leading tip 120 toward the cochlea 700, the cochlear implantable catheter 1 penetrates into the cochlea 700 through a round window membrane 710 of the cochlea 700. The leading tip 120 of the cochlear implantable catheter 1 at least reaches a position that is close to scala tympani at the innermost part of the cochlea 700.

The proximal end 110 of the implantable catheter body 100 of the cochlear implantable catheter 1 is connected to the delivery module 200 of the cell delivery device 2 via the connection tube 400. Through the delivery module 200, the fluid that contains the cells or the medications for treating hearing loss is delivered into the implantable catheter body 100 of the cochlear implantable catheter 1. Then, said fluid flows from the central lumen 130 to the lateral through holes 150, and enters the cochlea 700 of the patient through the lateral through holes 150.

Since the lateral through holes 150 of the cochlear implantable catheter 1 are arranged within a length range of the implant section of the implantable catheter body 100, the pressure of the fluid is prevented from concentrating on a single positon within the cochlea 700. In addition, the cells or the medications for treating hearing loss can be evenly distributed at different positions within the cochlea 700, thereby improving a treatment effect.

It should be noted that, in the embodiment shown in FIG. 8, cells 800 contained in the fluid that is delivered by the cochlear implantable catheter 1 are in the form of the cell spheroids (which are formed by a combination of cells). However, the present disclosure is not limited thereto. For example, the cells 800 contained in the fluid can also be a singular cell or cellular growth factor.

Furthermore, when the fluid is being delivered into the cochlea 700 of the patient by the cell delivery device 2, controlling a speed and the flow rate of the fluid and controlling the pressure of the fluid are both required. On one hand, the fluid that contains the cells 800 or the medications can be prevented from leaking from the cochlea 700. On the other hand, death or damage to the cells 800 within the fluid due to bearing an overly large pressure (which is caused by the pressure of the fluid being too great) can be prevented.

In one exemplary embodiment of the present disclosure, the cell delivery device 2 is configured to be capable of delivering the fluid having a delivery viscosity of between 2 Pa·s and 5 Pa·s through the cochlear implantable catheter 1 at a flow rate of between 0.1 sccm and 0.5 sccm, and the pressure of the fluid ranges between 1,000 MPa and 1,500 MPa.

It is worth mentioning that the cochlear implantable catheter 1 of the present disclosure is designed to be taken out of the cochlea 700 of the patient right after the fluid that contains the cells or the medications for treating hearing loss is delivered into the cochlea 700, and is not continuously implanted into the cochlea 700 of the patient. As such, after the cells or the medications are delivered into the cochlea 700 of the patient and the cochlear implantable catheter 1 is taken out, the operator can choose whether to seal the round window membrane 710 of the cochlea 700 (for keeping the cells or the medications within the cochlea 700) or implant the electrodes of the cochlear implant before sealing the cochlea 700 according to therapeutic requirements.

### Second Embodiment

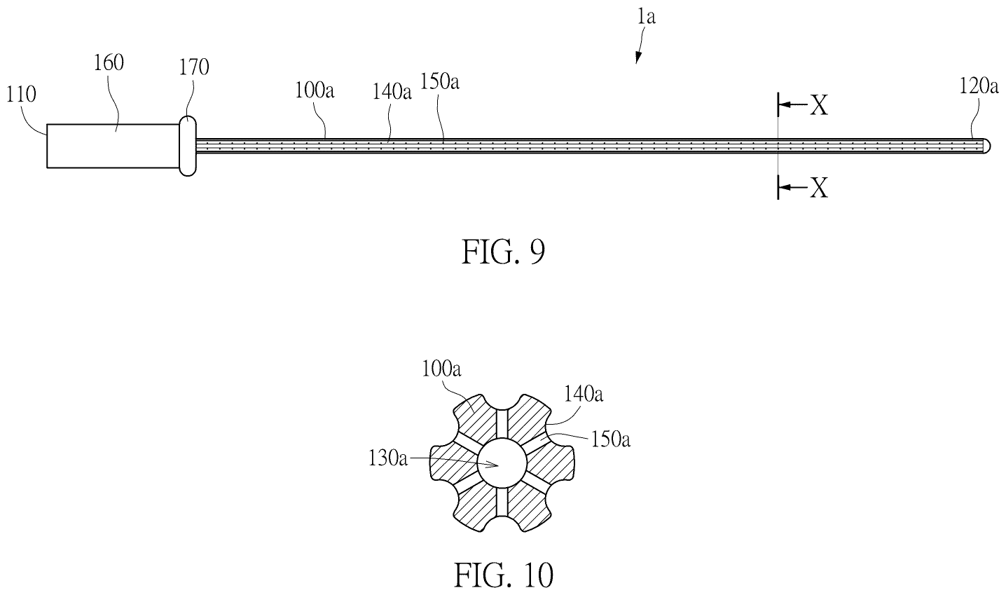

Reference is made to FIG. 9 and FIG. 10, in which a second embodiment of the cochlear implantable catheter of the present disclosure is shown. The technical features of the present embodiment are similar to those of the first embodiment, and the similarities therebetween will not be reiterated herein.

As shown in FIG. 9, in the present embodiment, a cochlear implantable catheter 1a has a plurality of guide grooves 140a formed on an outer surface of an implant section of an implantable catheter body 100a. As linear grooves formed on the outer surface of the implant section of the implantable catheter body 100a, the guide grooves 140a are arranged along a path that is parallel to a central axis of the implantable catheter body 100a and extend in a longitudinal direction of the implantable catheter body 100a.

From a cross-section of the implant section of the implantable catheter body 100a (as shown in FIG. 10), it can be observed that the guide grooves 140a are arranged to be equiangularly spaced apart from each other and surround an outer side of the implantable catheter body 100a. In addition, the guide grooves 140a are recessed in a direction from an outer surface of the implantable catheter body 100a to a center of the implantable catheter body 100a. The guide grooves 140a can be used to increase bending elasticity of the implantable catheter body 100a. When the implantable catheter body 100a enters the cochlea, the guide grooves 140a can generate the flow-guiding effect to decrease resistance from the implantable catheter body 100a and the lymph in the cochlea, thereby achieving the effect of reducing the lymphatic extravasation.

### Beneficial Effects of the Embodiments

In conclusion, in the cochlear implantable catheter and the cell delivery device provided by the present disclosure, the cochlear implantable catheter is designed to be easily implanted into the cochlea of the patient and have good bendability. The cochlear implantable catheter can easily bend and enter deep into the cochlear duct in a spiral manner (along the spiral ligament on the outer wall of the cochlear duct). Through the design of the guide groove, the resistance from the outer wall of the implantable catheter body and the lymph in the cochlea can be decreased, thereby increasing an implant success rate of the cochlear implantable catheter and preventing damage to the cochlea.

Furthermore, since the cochlear implantable catheter of the present disclosure is designed to have the guide groove and the lateral through holes that are evenly arranged on the implantable catheter body, the cells or the medications can evenly reach different positions of the cochlea, and the lymphatic extravasation of the cochlea can be prevented.

The foregoing description of the exemplary embodiments of the disclosure has been presented only for the purposes of illustration and description and is not intended to be exhaustive or to limit the disclosure to the precise forms disclosed. Many modifications and variations are possible in light of the above teaching.

The embodiments were chosen and described in order to explain the principles of the disclosure and their practical application so as to enable others skilled in the art to utilize the disclosure and various embodiments and with various modifications as are suited to the particular use contemplated. Alternative embodiments will become apparent to those skilled in the art to which the present disclosure pertains without departing from its spirit and scope.

What is claimed is:

1. A cochlear implantable catheter, which is configured to deliver cells or medications to a cochlea of a patient, comprising:
an implantable catheter body, wherein the implantable catheter body is flexible, and a proximal end and a leading tip are defined at two opposite ends of the implantable catheter body in a longitudinal direction; wherein a portion of the implantable catheter body that is implanted into the cochlea of the patient is defined as an implant section, the implantable catheter body has a connection end that is disposed at one side of the implant section toward the proximal end, a central lumen that penetrates from a center of the proximal end to the leading tip is formed inside the implantable catheter body, and the implant section has at least one guide groove formed on an outer surface thereof and a plurality of lateral through holes that penetrate from the outer surface of the implant section to the central lumen;
wherein the implantable catheter body is configured to penetrate into the cochlea of the patient by orienting the leading tip toward the cochlea of the patient, and is capable of bending and moving forward along a spiral structure of the cochlea of the patient, so as to enter between a basal turn and an apical turn of the cochlea of the patient; wherein the cells or the medications are capable of being guided into the central lumen from the proximal end and entering the cochlea through the central lumen and the plurality of lateral through holes;
wherein the at least one guide groove is a spiral groove formed on the outer surface of the implant section of the implantable catheter body along a spiral path that extends in the longitudinal direction of the implantable catheter body, or is a groove formed along a central axial path of the implantable catheter body and extending in the longitudinal direction of the implantable catheter body, such that the implantable catheter body is bendable when penetrating into the cochlea of the patient, and a flow-guiding effect is generated to reduce lymphatic extravasation of the cochlea.

2. The cochlear implantable catheter according to claim 1, wherein a length of the implant section of the implantable catheter body is configured to range between 8 mm and 30 mm, and a diameter of the implant section ranges between 0.3 mm and 1.0 mm.

3. The cochlear implantable catheter according to claim 2, wherein a diameter of the central lumen is configured to range between 300 μm and 500 μm, and a diameter of the plurality of lateral through holes is configured to range between 50 μm and 300 μm.

4. The cochlear implantable catheter according to claim 2, wherein a diameter of the central lumen is configured to range between 100 μm and 200 μm, and a diameter of the plurality of lateral through holes is configured to range between 10 μm and 50 μm.

5. The cochlear implantable catheter according to claim 2, wherein a protruding ring is disposed at a junction of the connection end and the implant section of the implantable catheter body, and a diameter of the protruding ring is greater than the diameter of the implant section of the implantable catheter body and a diameter of the connection end of the implantable catheter body.

6. The cochlear implantable catheter according to claim 1, wherein the at least one guide groove is limited to being the spiral groove, and a helix angle of the at least one guide groove is configured to range between 15 degrees and 60 degrees.

7. The cochlear implantable catheter according to claim 1, wherein the at least one guide groove is limited to being a linear groove; wherein, when a quantity of the at least one guide groove is plural, the guide grooves are arranged to be equiangularly spaced apart on an outer side of the implantable catheter body.

8. The cochlear implantable catheter according to claim 1, wherein a groove depth of the at least one guide groove is configured to range between 10 μm and 200 μm.

9. A cell delivery device, which is configured to deliver cells or medications to a cochlea of a patient, comprising:
a cochlear implantable catheter, wherein the cochlear implantable catheter includes an implantable catheter body, the implantable catheter body is flexible, and a proximal end and a leading tip are defined at two opposite ends of the implantable catheter body in a longitudinal direction; wherein a portion of the implantable catheter body that is implanted into the cochlea of the patient is defined as an implant section, the implantable catheter body has a connection end that is disposed at one side of the implant section toward the proximal end, a central lumen that penetrates from a center of the proximal end to the leading tip is formed inside the implantable catheter body, and the implant section has at least one guide groove formed on an outer surface thereof and a plurality of lateral through holes that penetrate from the outer surface of the implant section to the central lumen;
wherein the implantable catheter body is configured to penetrate into the cochlea of the patient by orienting the leading tip toward the cochlea of the patient, and is capable of bending and moving forward along a spiral structure of the cochlea of the patient, so as to enter between a basal turn and an apical turn of the cochlea of the patient; wherein the cells or the medications are capable of being guided into the central lumen from the proximal end and entering the cochlea through the central lumen and the plurality of lateral through holes;
wherein the at least one guide groove is a spiral groove formed on the outer surface of the implant section of the implantable catheter body along a spiral path that extends in the longitudinal direction of the implantable catheter body, or is a groove formed along a central axial path of the implantable catheter body and extending in the longitudinal direction of the implantable catheter body, such that the implantable catheter body is bendable when penetrating into the cochlea of the patient, and a flow-guiding effect is generated to reduce lymphatic extravasation of the cochlea; and
a delivery module connected to the cochlear implantable catheter, wherein the delivery module includes a pressure generator and a connection tube connected to an outlet of the pressure generator; wherein a fluid that contains the cells or the medications is stored in the pressure generator, and the pressure generator drives and outputs the fluid that contains the cells or the medications from the outlet to the connection tube; wherein one end of the connection tube that is away from the pressure generator is connected to the proximal end of the implantable catheter body, such that the cells or the medications are capable of entering the central lumen of the implantable catheter body and entering the cochlea of the patient through the central lumen and the plurality of lateral through holes.

10. The cell delivery device according to claim 9, wherein the delivery module is configured to be capable of delivering the fluid having a delivery viscosity of between 2 Pa·s and 5 Pa·s through the cochlear implantable catheter at a flow rate of between 0.1 sccm and 0.5 sccm, and a pressure of the fluid ranges between 1,000 MPa and 1,500 MPa.

11. The cell delivery device according to claim 9, wherein a length of the implant section of the implantable catheter body is configured to range between 8 mm and 30 mm, and a diameter of the implant section ranges between 0.3 mm and 1.0 mm.

12. The cell delivery device according to claim 11, wherein a diameter of the central lumen is configured to range between 300 μm and 500 μm, and a diameter of the plurality of lateral through holes is configured to range between 50 μm and 300 μm.

13. The cell delivery device according to claim 11, wherein a diameter of the central lumen is configured to range between 100 μm and 200 μm, and a diameter of the plurality of lateral through holes is configured to range between 10 μm and 50 μm.

14. The cell delivery device according to claim 11, wherein a protruding ring is disposed at a junction of the connection end and the implant section of the implantable catheter body, and a diameter of the protruding ring is greater than the diameter of the implant section of the implantable catheter body and a diameter of the connection end of the implantable catheter body.

15. The cell delivery device according to claim 9, wherein the at least one guide groove is limited to being the spiral groove, and a helix angle of the at least one guide groove is configured to range between 15 degrees and 60 degrees.

16. The cell delivery device according to claim 9, wherein the at least one guide groove is limited to being a linear groove; wherein, when a quantity of the at least one guide groove is plural, the guide grooves are arranged to be equiangularly spaced apart on an outer side of the implantable catheter body.

17. The cell delivery device according to claim 9, wherein a groove depth of the at least one guide groove is configured to range between 10 μm and 200 μm.
